(19) United States
(12) Patent Application Publication
(10) Pub. No.: US 2018/0235675 A1
(43) Pub. Date: Aug. 23, 2018

(54) NOTCH TABS FOR POSITIONING RODS IN SCREW HEADS

(71) Applicants: Harry LOCKSTADT, Lexington, KY (US); Troy M. WALTERS, Plymouth, IN (US)

(72) Inventors: Harry LOCKSTADT, Lexington, KY (US); Troy M. WALTERS, Plymouth, IN (US)

(21) Appl. No.: 15/902,274

(22) Filed: Feb. 22, 2018

Related U.S. Application Data
(60) Provisional application No. 62/462,004, filed on Feb. 22, 2017.

(51) Int. Cl. A61B 17/70

(52) U.S. Cl. CPC A61B 17/7083; A61B 2090/037; A61B 17/7032

(57) ABSTRACT

A tab is removably connected to a surgical screw having a head. The tab has a longitudinal slot for receiving a connecting rod therethrough for connection to the screw head. The tab has a notch for enlarging a portion of the slot to facilitate the passage of the rod through the tab. A plurality of tabs can be aligned to enable a connecting rod to pass therethrough and be connected to a plurality of screw heads. The length of the enlarged slot portion is less than the length of the longitudinal slot.

### CROSS-REFERENCES TO RELATED APPLICATIONS

This application claims the priority of Provisional Patent Application No. 62/462,004 filed on Feb. 22, 2017 and entitled “NOTCH TABS FOR POSITIONING RODS IN SCREW HEADS”.

### BACKGROUND OF THE INVENTION

### Field of the Invention

The present invention relates to a surgical technique for a spinal system wherein screws with U-shaped heads having openings with internal threads are positioned in the spine. A connecting rod is positioned in the openings of the screw heads and is retained therein by set screws that are threaded into the screw heads.

### Description of the Background Art

In a cannulated screw system, the screws 10 have towers or tabs 22 removably connected thereto to allow surgery to be completed by making only stab incisions for the placement of each screw 10. The towers or tabs 22 have elongated longitudinal slots 24 for receiving a connecting rod 18 and guiding it into the screw heads 12. If the towers or tabs 22 are not in the same rotated position, it will be difficult to insert the connecting rod 18 therein because the aligned slots 24 will be narrowed. This problem is solved by the present invention.

### BRIEF SUMMARY OF THE INVENTION

In accordance with the present invention, a notch is formed in the tab to enlarge a portion of the width of the longitudinal slot. Minimally invasive type approaches to spine surgery include either towers attached to pedicle screws or long break-off tabs to allow the surgery to be completed with only making stab incisions for the placement of each screw. One of the more challenging parts of this surgery is placing a connecting rod in the correct position at the bottom of the screw heads without trapping tissue. The rods are fed down elongated slots in the tabs and then rotated to pass thru the screw head in the next level. The more levels being fused requires the rod to be passed thru each adjacent screw head. Since the normal rod slot is only slightly larger in width than the rod diameter, the increased width and reduced length in the notch opening of the tab slot provides a larger target to pass the rod through. It also addresses the issue of what occurs when the head of the screw is slightly rotated from the long axis of the rod. Since the slot width in the screw is near the diameter of the rod, a small rotation decreases the slot width that is open to the rod. With the notch added, the issue of slight rotation is greatly lessened because the width of the slot opening is larger and the length of the enlarged slot is shorter.

### BRIEF DESCRIPTION OF THE DRAWINGS

FIG. 1 is a perspective view of a surgical screw used in the present invention;

FIG. 2 is a perspective view of a set screw used with the surgical screw shown in FIG. 1;

FIG. 3 is a perspective view of the head of a surgical screw with a connecting rod extending through the head and retained therein by a set screw;

FIG. 4 is a perspective view of a plurality of elongated tabs mounted on a pedicle with a connecting rod being inserted through the tabs;

FIG. 5 is a perspective view of one side of a conventional tab;

FIG. 6 is a perspective view of the tab of FIG. 5 after some rotation on its longitudinal axis;

FIG. 7 is a perspective view of one side of a new and improved tab of the present invention;

FIG. 8 is a perspective view of another side of the tab of FIG. 7;

FIG. 9 is a perspective view of a portion of the tab of FIG. 7 after some rotation on its longitudinal axis; and

FIGS. 10, 11 and 12 are perspective views of another embodiment of the tab of the present invention.

### DETAILED DESCRIPTION OF THE INVENTION

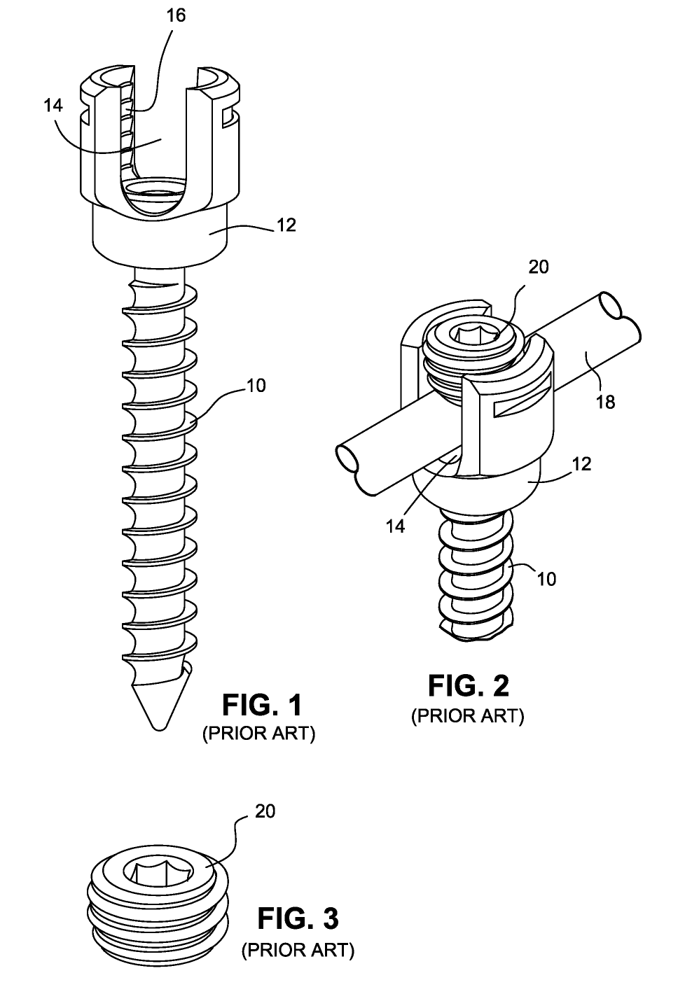

For a better understanding of the present invention, it relates to surgical techniques for a spinal system wherein screws 10 with U-shaped heads 12 having openings 14 with internal threads 16 are positioned in the spine, as shown in FIG. 1. A connecting rod 18 is positioned in the openings 14 of the screw heads 12 and is retained therein by set screws 20 that are threaded into the screw heads 12, as shown in FIGS. 2 and 3. Because the screw head openings 14 are not much larger in width than the diameter of the connecting rod 18, it may be difficult to position a connecting rod 18 in a number of screw heads 12 if their openings 14 are not properly aligned.

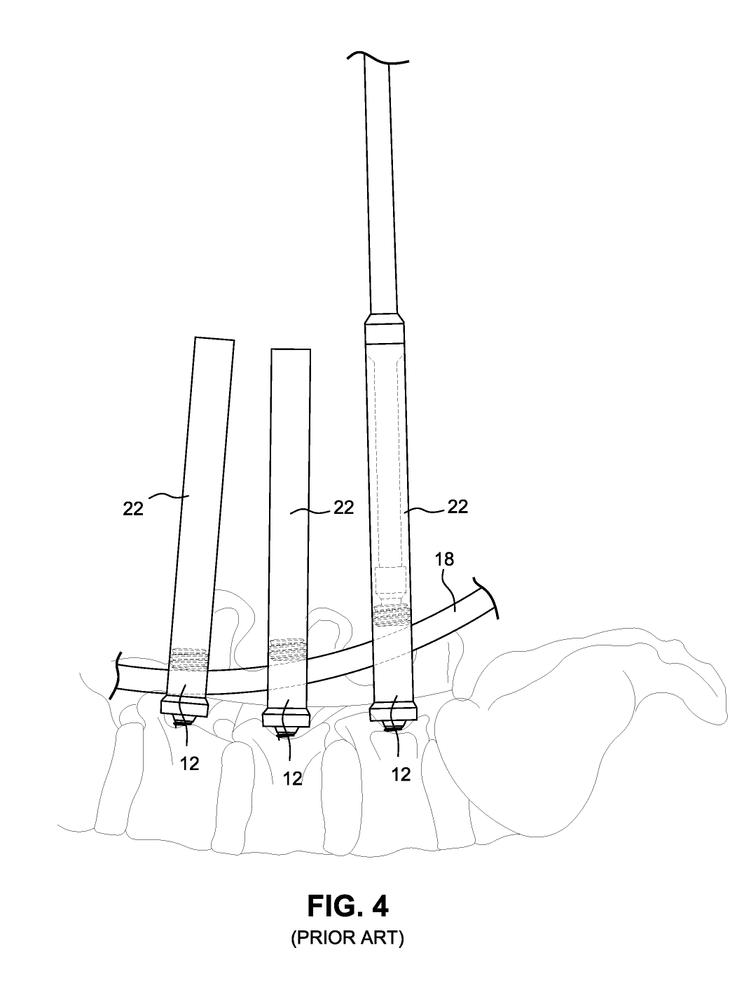

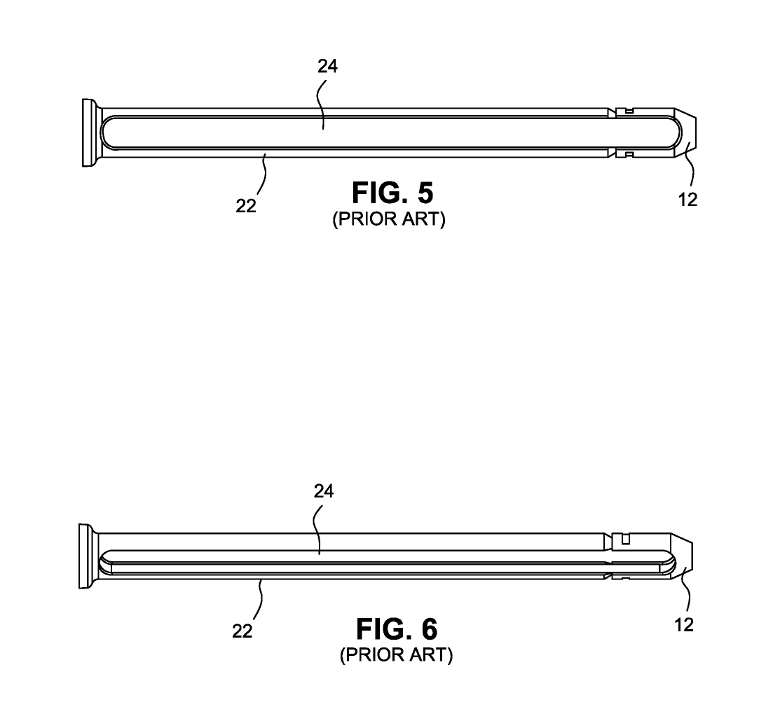

FIGS. 5 and 6 illustrate a conventional break off tab 22 for a pedicle screw having a head 12. The tab 22 has a longitudinal slot 24 for receiving a connecting rod 18 and guiding it into the screw head 12. If a plurality of tabs 22 shown in FIG. 4 are not in the same rotated position, it will be difficult to insert the connecting rod 18 therein because the aligned slots 24 will be narrowed as shown in FIG. 6.

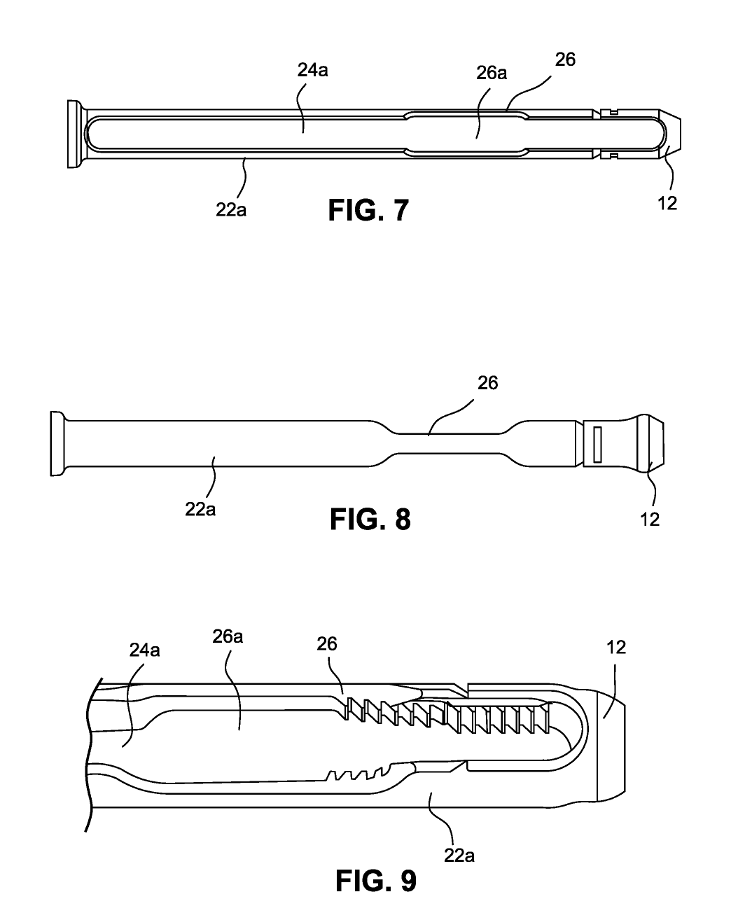

As shown in FIGS. 7-9, the new and improved tab 22a of the present invention is provided with a notch 26 for enlarging the width of a portion 26a of the slot 24a of the tab 22a. The enlarged portion 26a of the slot 24a is of reduced length and provides a larger target for the insertion of the connecting rod 18. Also, if the head 12 of the screw is slightly rotated from the long axis of the rod, the enlarged portion 26a of the slot 24a and its shorter length facilitates the passage of the rod, as shown in FIG. 9.

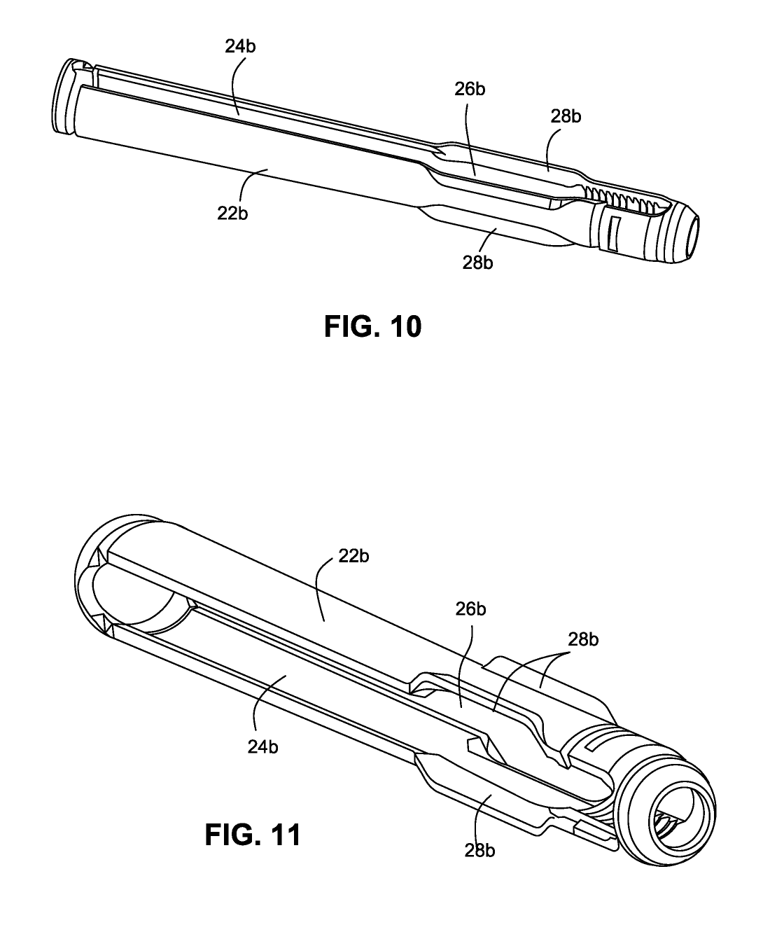

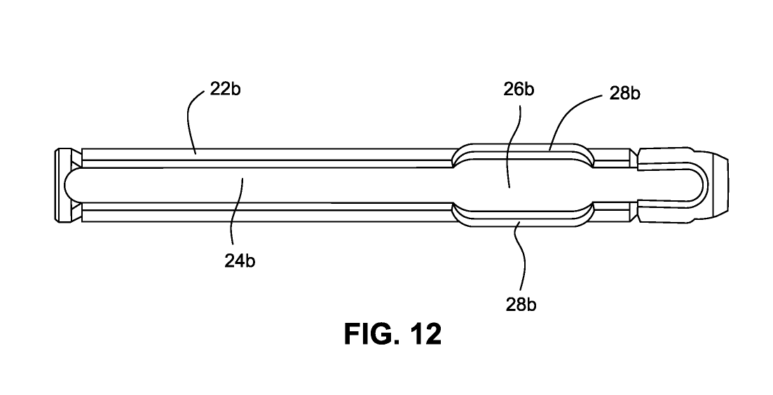

FIGS. 10-12 illustrate another embodiment of the present invention with a modified tab 22b having a notch 26b for enlarging the slot 24b. The notch 26b is surrounded by lateral flanges 28b which serve to guide the connecting rod into the notch 26b.

1. A tab removably connected to a surgical screw having a head, the tab having a longitudinal slot for receiving a connecting rod therethrough for connection to the screw head, the tab having a notch for enlarging the width of a portion of the slot to facilitate the passage of the rod through the tab.

2. The tab of claim 1 having a reduced thickness in an area of the enlarged portion of the slot.

3. The tab of claim 1 having a lateral flange adjacent to the enlarged portion of the slot for guiding the connecting rod into the enlarged portion of the slot.

4. The tab of claim 3 having a plurality of lateral flanges for guiding the connecting rod into the enlarged portion of the slot.

5. The tab of claim 1 wherein the length of the enlarged slot portion is less than the length of the longitudinal slot.

6. A plurality of tabs removably connected to surgical screws, each having a head, the tabs having aligned longitudinal slots for receiving a connecting rod therethrough for connection to the screw heads, the tabs having aligned notches defining portions of the slots of enlarged width to facilitate the passage of the rod through the aligned slots of the tabs.

7. The tabs of claim 6 wherein the enlarged portions of the slots are of a length less than the length of the longitudinal slots.
